SEQUENCE LISTING (15 sequences)

SEQ ID NO: 1 - protein, 4 aa, Artificial Sequence
  Synthetic peptide
  MISC_FEATURE (1)..(1): Xaa can be any natural or non-canonical amino acid
  MISC_FEATURE (4)..(4): Xaa can be any natural or non-canonical amino acid
XCHX

SEQ ID NO: 2 - protein, 6 aa, Artificial Sequence
  Synthetic peptide
GCHGGR

SEQ ID NO: 3 - protein, 5 aa, Artificial Sequence
  Synthetic peptide
  MISC_FEATURE (1)..(1): Xaa can be any natural or non-canonical amino acid
  MISC_FEATURE (3)..(3): Xaa can be any natural or non-canonical amino acid
  MISC_FEATURE (5)..(5): Xaa can be any natural or non-canonical amino acid
XCXCX

SEQ ID NO: 4 - protein, 6 aa, Artificial Sequence
  Synthetic peptide
  MISC_FEATURE (1)..(1): Xaa can be any natural or non-canonical amino acid
  MISC_FEATURE (3)..(3): Xaa can be any natural or non-canonical amino acid
  MISC_FEATURE (6)..(6): Xaa can be any natural or non-canonical amino acid
XCXCHX

SEQ ID NO: 5 - protein, 9 aa, Artificial Sequence
  Synthetic peptide
QWGCGGCHG

SEQ ID NO: 6 - protein, 11 aa, Artificial Sequence
  Synthetic peptide
  MISC_FEATURE (3)..(3): Xaa can be any natural or non-canonical amino acid
  MISC_FEATURE (8)..(8): Xaa can be any natural or non-canonical amino acid
VQXCAQCXTVE

SEQ ID NO: 7 - protein, 11 aa, Artificial Sequence
  Synthetic peptide
VQKCAQCHTVE

SEQ ID NO: 8 - protein, 11 aa, Artificial Sequence
  Synthetic peptide
VQECAQCHTVE

SEQ ID NO: 9 - protein, 11 aa, Artificial Sequence
  Synthetic peptide
VQKCAQCMTVE

SEQ ID NO: 10 - protein, 11 aa, Artificial Sequence
  Synthetic peptide
VQHCAQCHTVE

SEQ ID NO: 11 - protein, 6 aa, Artificial Sequence
  Synthetic peptide
RCHGGR

SEQ ID NO: 12 - protein, 6 aa, Artificial Sequence
  Synthetic peptide
GCHGGD

SEQ ID NO: 13 - protein, 9 aa, Artificial Sequence
  Synthetic peptide
QHGCGGCHG

SEQ ID NO: 14 - protein, 10 aa, Artificial Sequence
  Synthetic peptide
QHGCGGCGHG

SEQ ID NO: 15 - protein, 10 aa, Artificial Sequence
  Synthetic peptide
QHGGCGGCHG

(19) United States
(12) Patent Application Publication
(10) Pub. No.: US 2020/0157188 A1
(43) Pub. Date: May 21, 2020

(54) SYNTHETIC HEME-CONTAINING MOLECULES AND THEIR USE

(71) Applicant: University of Pittsburgh - Of The Commonwealth System of Higher Education, Pittsburgh, PA (US)

(72) Inventors: Jason J. Rose, Pittsburgh, PA (US); Jesus Tejero Bravo, Pittsburgh, PA (US); Kazi R. Islam, Cheswick, PA (US); Mark T. Gladwin, Pittsburgh, PA (US); Raymond B. Yurko, McMurray, PA (US); Anthony W. DeMartino, Pittsburgh, PA (US)

(73) Assignee: University of Pittsburgh - Of the Commonwealth System of Higher Education, Pittsburgh, PA (US)

(21) Appl. No.: 16/627,167

(22) PCT Filed: Jun. 28, 2018
(86) PCT No.: PCT/US2018/040093

§ 371 Date: Dec. 27, 2019

Related U.S. Application Data
(60) Provisional application No. 62/525,909, filed on Jun. 28, 2017.

(51) Int. Cl. C07K 14/805; A61K 9/00

(52) U.S. Cl. CPC C07K 14/805; A61K 38/00; A61K 9/0019

(57) ABSTRACT

Synthetic heme-containing molecules are described. The heme-containing molecules include a heme group bound to either two non-contiguous peptides or a single contiguous peptide via cysteine residues. Use of the synthetic heme-containing molecules, such as for the treatment of carboxyhemoglobinemia, cyanide poisoning and hydrogen sulfide (H₂S) poisoning, is further described.

### CROSS REFERENCE TO RELATED APPLICATIONS

This application claims the benefit of U.S. Provisional Application No. 62/525,909, filed Jun. 28, 2017, which is herein incorporated by reference in its entirety.

### ACKNOWLEDGMENT OF GOVERNMENT SUPPORT

This invention was made with government support under grant numbers ES027390, HL125886 and HL132539, awarded by the National Institutes of Health. The government has certain rights in the invention.

### FIELD

This disclosure concerns novel synthetic heme-bound peptides that bind carbon monoxide with high affinity, and their use for the treatment of carboxyhemoglobinemia, cyanide poisoning and hydrogen sulfide poisoning.

### BACKGROUND

Inhalation exposure to carbon monoxide represents a major cause of environmental poisoning. Individuals can be exposed to carbon monoxide in the air under a variety of different circumstances, such as house fires, generators or outdoor barbeque grills used indoors, or during suicide attempts in closed spaces. Carbon monoxide binds to hemoglobin and to hemoproteins in cells, in particular the enzymes of the respiratory transport chain. The accumulation of carbon monoxide bound to hemoglobin and other hemoproteins impairs oxygen delivery and oxygen utilization for oxidative phosphorylation. This ultimately results in severe hypoxic and ischemic injury to vital organs such as the brain and the heart. Individuals who accumulate greater than 10% carbon carboxyhemoglobin in their blood are at risk for brain injury and neurocognitive dysfunction. Patients with very high carboxyhemoglobin levels typically suffer from irreversible brain injury, respiratory failure and/or cardiovascular collapse.

Despite the availability of methods to rapidly diagnose carbon monoxide poisoning with standard arterial and venous blood gas analysis and co-oximetry, and despite an awareness of risk factors for carbon monoxide poisoning, there are currently no available antidotes for this toxic exposure. The current therapy is to give 100% oxygen by face mask, and when possible, to expose patients to hyperbaric oxygen. The mechanism behind hyperbaric oxygen therapy is the oxygen will increase the rate of release of the carbon monoxide from hemoglobin and from tissues and accelerate the natural clearance of carbon monoxide. However, this therapy has only a modest effect on carbon monoxide clearance rates, and based on the complexity of hyperbaric oxygen facilities, this therapy is not available in the field and is often associated with significant treatment delays and transportation costs.

### SUMMARY

A need exists for an effective, rapid and readily available therapy to treat carboxyhemoglobinemia, cyanide poisoning and hydrogen sulfide poisoning. It is disclosed herein that synthetic heme-containing molecules, bound to either two separate peptides or a single contiguous peptide, are capable of binding CO with high affinity and displacing CO from hemoglobin, thereby acting as CO scavengers.

Provided herein are novel synthetic heme-containing molecules. In some embodiments, the synthetic heme-containing molecules of the present invention include a heme group bound to two non-contiguous peptides each having an amino acid sequence of the formula (X)₁₋₂₀C(X)₁₋₂₀ where X is any natural or non-canonical amino acid, wherein C represents a cysteine and the cysteine residue of each peptide is bound to the heme group. In other embodiments, the heme-containing molecule includes a single contiguous peptide having the formula (X)₁₋₂₀C(X)₁₋₅C(X)₁₋₂₀ (SEQ ID NO: 3) where X is any natural or non-canonical amino acid, wherein C represents a cysteine and the two cysteine residues of the peptide are bound to the heme group. In some embodiments, the peptide(s) includes at least one modification, such as an N-terminal and/or C-terminal modification.

Also provided herein is a method of removing carbon monoxide from hemoglobin in blood or animal tissue by contacting the blood or animal tissue with a synthetic heme-containing molecule disclosed herein. In some embodiments, the method is an in vitro method. In other embodiments, the method is an in vivo method in which contacting the blood or animal tissue with the synthetic heme-containing molecule includes administering a therapeutically effective amount of a synthetic heme-containing molecule to a subject.

Further provided are methods of treating carboxyhemoglobinemia, cyanide poisoning or hydrogen sulfide (H₂S) poisoning in a subject by administering to the subject a synthetic heme-containing molecule disclosed herein.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1B are schematics of a heme molecule bound via thioether bonds to two peptides (a “sandwich” configuration), which are represented by the top and bottom cylinders. The peptide sequences are of the formula (X)₁₋₂₀C(X)₁₋₂₀ (FIG. 1A) or (X)₁₋₂₀CH(X)₁₋₁₉ (SEQ ID NO: 1; FIG. 1B).

FIGS. 2A-2B are schematics of a heme molecule bound to a single peptide via two thioether bonds (a “pacman” configuration). The peptide is represented by the C-shaped cylinder. The sequence of the peptide is of the formula (X)₁₋₂₀C(X)₁₋₅C(X)₁₋₂₀ (SEQ ID NO: 3; FIG. 2A) or (X)₁₋₂₀C(X)₁₋₅CH(X)₁₋₁₉ (SEQ ID NO: 4; FIG. 2B).

FIG. 3 shows reference spectra of oxidized state, deoxy-state and carboxy-state of microperoxidase-11 (top left), a synthetic heme molecule bound to two peptide chains each having the formula Ac-GCHGGR (SEQ ID NO: 2) wherein “Ac” indicates an acetylation of the glycine residue (top right), and a synthetic heme molecule bound to a single contiguous peptide chain of the formula Ac-QHGCGGCHG (SEQ ID NO: 13) that is bound to the heme group via two thioether bonds and wherein “Ac” indicates an acetylation of the glutamine residue (bottom).

FIG. 4 shows ultraviolet (UV)-visible spectra kinetic curves for 100% CO bound hemoglobin (HbCO) mixed with reduced microperoxidase-11 (MP11) (left) and spectral changes during the reaction of MP11 with CO-bound Hb reveal a k_{obs} for CO scavenging by MP11 of k₁=0.0215 s-1 (right).

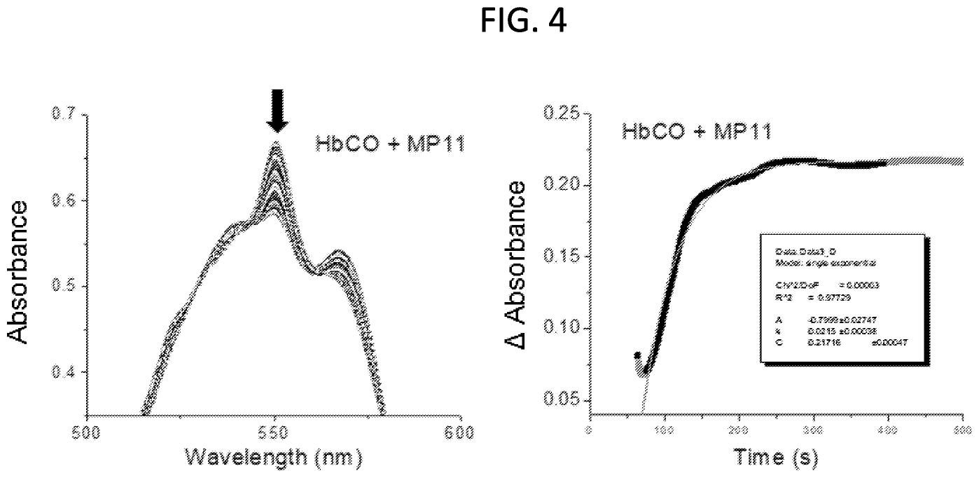

FIG. 5 shows UV-visible spectra kinetic curves for 100% CO bound hemoglobin (HbCO) mixed with reduced heme-bound QHGCGGCHG (SEQ ID NO: 13) peptide (left) and spectral changes during the reaction of reduced heme-bound QHGCGGCHG (SEQ ID NO: 13) peptide with CO-bound Hb reveal a k, for CO scavenging by QHGCGGCHG peptide of k₁=0.18282 s-1 and k₂=0.00968 s-1 (right).

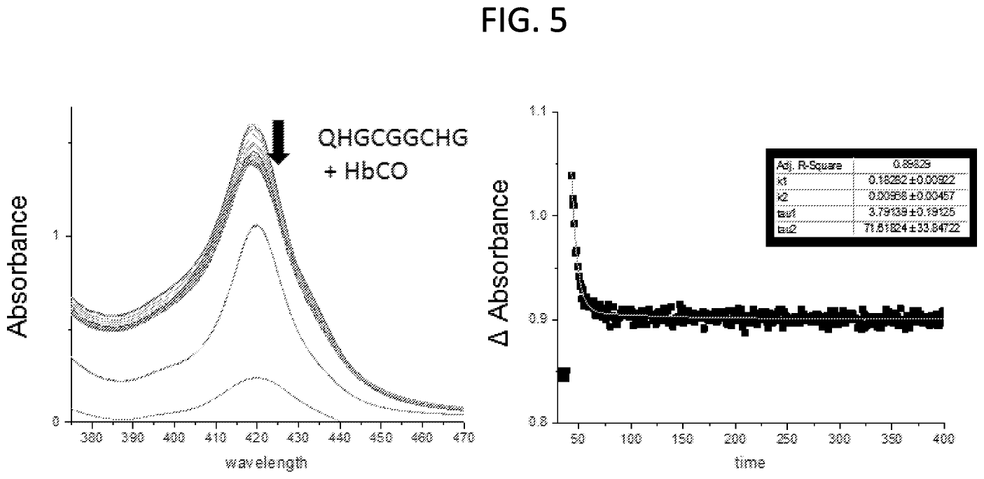

FIG. 6 shows UV-visible spectra kinetic curves for 100% CO bound hemoglobin (HbCO) mixed with reduced heme-bound RCHGGR (SEQ ID NO: 11) peptides (two non-contiguous peptides) (left) and spectral changes during the reaction of reduced heme-bound RCHGGR (SEQ ID NO: 11) peptides with CO-bound Hb reveal a k, for CO scavenging by RCHGGR peptide of k₁=0.02576 s-1 and k₂=0.01241 s-1 (right).

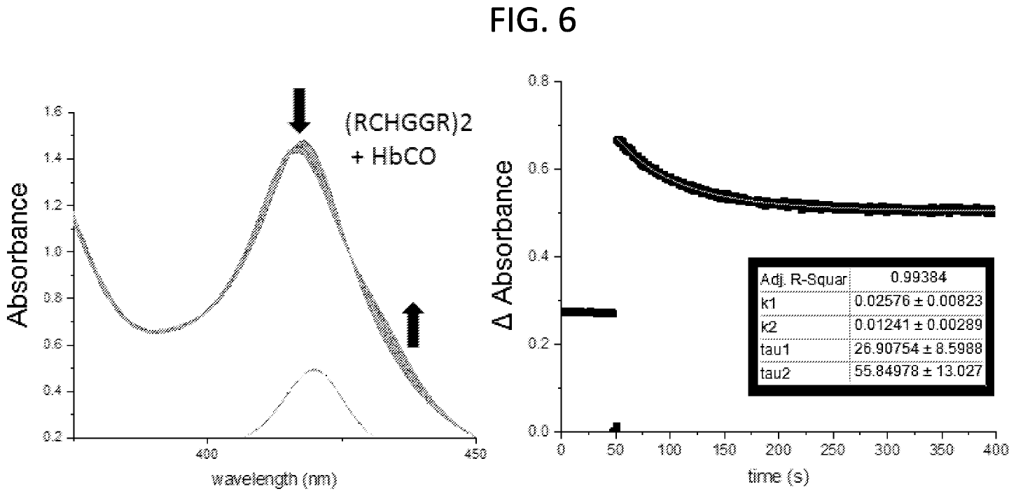

FIG. 7 shows the structure of heme A, heme B, heme C, heme D and heme O.

### SEQUENCE LISTING

The amino acid sequences listed in the accompanying Sequence Listing are shown using standard three letter code for amino acids, as defined in 37 C.F.R. 1.822. The Sequence Listing is submitted as an ASCII text file, created on Jun. 28, 2018, 4.06 KB, which is incorporated by reference herein. In the accompanying sequence listing:

SEQ ID NO: 1 is an amino acid motif of a peptide capable of binding a heme molecule via a single bond.

SEQ ID NO: 2 is the amino acid sequence of a representative peptide that binds a heme molecule via a single bond.

SEQ ID NO: 3 is an amino acid motif of a peptide capable of binding a heme molecule via two bonds.

SEQ ID NO: 4 is an amino acid motif of a peptide capable of binding a heme molecule via two bonds.

SEQ ID NO: 5 is the amino acid sequence of a representative peptide that binds a heme molecule via two bonds.

SEQ ID NO: 6 is the amino acid sequence of a representative peptide that binds a heme molecule via two bonds.

SEQ ID NO: 7 is the amino acid sequence of a representative peptide that binds a heme molecule via two bonds.

SEQ ID NO: 8 is the amino acid sequence of a representative peptide that binds a heme molecule via two bonds.

SEQ ID NO: 9 is the amino acid sequence of a representative peptide that binds a heme molecule via two bonds.

SEQ ID NO: 10 is the amino acid sequence of a representative peptide that binds a heme molecule via two bonds.

SEQ ID NO: 11 is the amino acid sequence of a representative peptide that binds a heme molecule via two bonds.

SEQ ID NO: 12 is the amino acid sequence of a representative peptide that binds a heme molecule via two bonds.

SEQ ID NO: 13 is the amino acid sequence of a representative peptide that binds a heme molecule via two bonds.

SEQ ID NO: 14 is the amino acid sequence of a representative peptide that binds a heme molecule via two bonds.

SEQ ID NO: 15 is the amino acid sequence of a representative peptide that binds a heme molecule via two bonds.

### DETAILED DESCRIPTION

### I. Abbreviations

CO carbon monoxide

H₂S hydrogen sulfide

Hb hemoglobin

HbCO carboxyhemoglobin

### II. Terms and Methods

Unless otherwise noted, technical terms are used according to conventional usage.

Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Administration: To provide or give a subject an agent, such as a therapeutic agent (e.g. a microperoxidase), by any effective route. Exemplary routes of administration include, but are not limited to, injection or infusion (such as subcutaneous, intramuscular, intradermal, intraperitoneal, intrathecal, intravenous, intracerebroventricular, intrastriatal, intracranial and into the spinal cord), oral, intraductal, sublingual, rectal, transdermal, intranasal, vaginal and inhalation routes.

Antidote: An agent that neutralizes or counteracts the effects of a poison.

Carbon monoxide (CO): A colorless, odorless and tasteless gas that is toxic to humans and animals when encountered at sufficiently high concentrations. CO is also produced during normal animal metabolism at low levels.

Carboxyhemoglobin (HbCO): A stable complex of carbon monoxide (CO) and hemoglobin (Hb) that forms in red blood cells when CO is inhaled or produced during normal metabolism.

Carboxyhemoglobinemia or carbon monoxide poisoning: A condition resulting from the presence of excessive amounts of carbon monoxide in the blood. Typically, exposure to CO of 100 parts per million (ppm) or greater is sufficient to cause carboxyhemoglobinemia. Symptoms of mild acute CO poisoning include lightheadedness, confusion, headaches, vertigo, and flu-like effects; larger exposures can lead to significant toxicity of the central nervous system and heart, and even death. Following acute poisoning, long-term sequelae often occur. Carbon monoxide can also have severe effects on the fetus of a pregnant woman. Chronic exposure to low levels of carbon monoxide can lead to depression, confusion, and memory loss. Carbon monoxide mainly causes adverse effects in humans by combining with hemoglobin to form carboxyhemoglobin (HbCO) in the blood. This prevents oxygen binding to hemoglobin, reducing the oxygen-carrying capacity of the blood, leading to hypoxia. Additionally, myoglobin and mitochondrial cytochrome oxidase are thought to be adversely affected. Carboxyhemoglobin can revert to hemoglobin, but the recovery takes time because the HbCO complex is fairly stable. Current methods of treatment for CO poisoning including administering 100% oxygen or providing hyperbaric oxygen therapy.

Contacting: Placement in direct physical association; includes both in solid and liquid form. When used in the context of an in vivo method, “contacting” also includes administering.

Cyanide poisoning: A type of poisoning that results from exposure to some forms of cyanide, such as hydrogen cyanide gas and cyanide salt. Cyanide poisoning can occur from inhaling smoke from a house fire, exposure to metal polishing, particular insecticides and certain seeds (such as apple seeds). Early symptoms of cyanide poisoning include headache, dizziness, rapid heart rate, shortness of breath and vomiting. Later symptoms include seizures, slow heart rate, low blood pressure, loss of consciousness and cardiac arrest.

Cytoglobin: A globin molecule that is ubiquitously expressed in all tissues. Cytoglobin is a hexacoordinate hemoglobin that has been reported to facilitate diffusion of oxygen through tissues, reduce nitrite to nitric oxide, and play a cytoprotective role in hypoxic conditions and under oxidative stress.

Globin: A heme-containing protein involved in the binding and/or transport of oxygen.

Globins include, for example, hemoglobin, myoglobin, neuroglobin and cytoglobin.

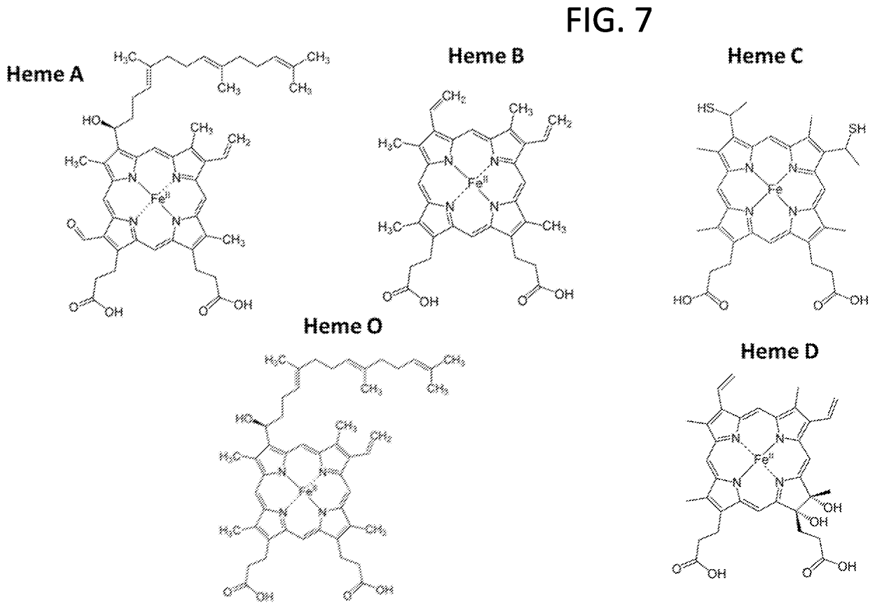

Heme: A cofactor consisting of a Fe²⁺ (ferrous) ion contained in the center of a porphyrin. A “heme protein” is a metalloprotein containing a heme prosthetic group. Heme-containing proteins include, but are not limited to, hemoglobin, myoglobin, cytoglobin, neuroglobin and cytochrome. In some embodiments, the term “heme” includes any natural or synthetic heme with a vinyl (—CH—CH₂) group. In yet other embodiments, the term “heme” includes porphyrins with vinyl groups in C3 and/or C8. In some embodiments, the term “heme” includes heme A, heme B, heme C, heme D, heme O, heme I, heme m, or heme S (FIG. 7; see e.g., Lin, Biochim Biophys Acta 1854(8):844-859, 2015; Ajioka et al., Biochim Biophys Acta 1763(7):723-736, 2006; Caughey et al., J Biol Chem 250:7602-7622, 1975; Kleingardner and Bren, Acc Chem Res 48(7):1845-1852, 2015; Bali et al., Cell Mol Life Sci 71(15):2837-2863, 2014; Cheesman et al., J Am Chem Soc 126: 4157-4166, 2004, each of which is herein incorporated by reference).

Hemoglobin (Hb): The iron-containing oxygen-transport metalloprotein in the red blood cells of the blood in vertebrates and other animals. In humans, the hemoglobin molecule is an assembly of four globular protein subunits. Each subunit is composed of a protein chain tightly associated with a non-protein heme group. Each protein chain arranges into a set of alpha-helix structural segments connected together in a globin fold arrangement, so called because this arrangement is the same folding motif used in other heme/globin proteins. This folding pattern contains a pocket which strongly binds the heme group.

Heterologous: A heterologous protein or polypeptide refers to a protein or polypeptide derived from a different source or species.

Hydrogen sulfide poisoning: A type of poisoning resulting from excess exposure to hydrogen sulfide (H₂S). H₂S binds iron in the mitochondrial cytochrome enzymes and prevents cellular respiration. Exposure to lower concentrations of H₂S can cause eye irritation, sore throat, coughing, nausea, shortness of breath, pulmonary edema, fatigue, loss of appetite, headaches, irritability, poor memory and dizziness. Higher levels of exposure can cause immediate collapse, inability to breath and death.

Isolated: An “isolated” biological component (such as a nucleic acid molecule, protein, or cell) has been substantially separated or purified away from other biological components in the cell, blood or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Microperoxidase (MP): A small peptide, having two cysteine residues, that is covalently bound to a porphyrin moiety. Microperoxidases are obtained from cytochrome c proteolysis or through artificial synthesis. One exemplary, and non-limiting, microperoxidase is MP11 which has a peptide sequence of VQKCAQCHTVE (SEQ ID NO: 7)

Myoglobin (Mb): A member of the globin family of proteins. Myoglobin is an iron- and oxygen-binding protein found in the muscle tissue of all vertebrates and nearly all mammals. In humans, myoglobin is only found in the bloodstream after muscle injury. Unlike hemoglobin, myoglobin contains only one binding site for oxygen (on the one heme group of the protein), but its affinity for oxygen is greater than the affinity of hemoglobin for oxygen.

Neuroglobin (Ngb): A member of the globin family of proteins. The physiological function of neuroglobin is currently unknown, but is thought to provide protection under hypoxic or ischemic conditions. Neuroglobin is expressed in the central and peripheral nervous system, cerebral spinal fluid, retina and endocrine tissues.

Non-canonical amino acid: Any amino acid that is not one of the 20 standard amino acids found in nature and directly encoded by the genetic code. “Non-canonical” amino acids are also referred to as “non-standard” or “unnatural” amino acids. In some embodiments, a non-canonical amino acid is a modified amino acid including but not limited to an amino acid with a modified C-terminal, modified N-terminal, or a combination thereof. In some embodiments, N-terminal modifications include but are not limited to formylation, acetylation, propionylation, pyroglutamate formation, myristoylation, palmitylation, S-palmitoylation, mono-methylation, di-methylation, or tri-methylation. In some embodiments, C-terminal modifications include but are not limited to methylation or alpha-amidation (see, e.g., Marino et al., ACS Chem Biol 10:1754-1764, 2015, which is incorporated herein by reference). In some embodiments, a non-canonical amino acid is a modified amino acid including but not limited to a methylated amino acid (i.e. a mono-, di-, and tri-methylated amino acid), an amino acid conjugated to a polyethylene glycol polymer, an amino acid conjugated to biotin, an amino acid conjugated to fluorescein isothiocyanate, an amino acid conjugated to a carrier protein (i.e. bovine serum albumin, ovalbumin, or keyhole limpet hemocyanin), a radioactive isotope (i.e. ²H, ¹⁵N, ¹³C, or both ¹⁵N and 1³C) labeled amino acid, or any combination thereof.

Peptide or Polypeptide: A polymer in which the monomers are amino acid residues which are joined together through amide bonds. When the amino acids are alpha-amino acids, either the L-optical isomer or the D-optical isomer can be used, the L-isomers being preferred. The terms “peptide,” “polypeptide” or “protein” as used herein are intended to encompass any amino acid sequence and include modified sequences. The terms “peptide” and “polypeptide” are specifically intended to cover naturally occurring proteins, as well as those which are recombinantly or synthetically produced. In some embodiments, the C-terminus of the peptides or polypeptides disclosed herein is modified. In some embodiments the C-terminus of the peptides or polypeptides disclosed herein is amidated. In some embodiments, the N-terminus of the peptides or polypeptides disclosed herein is modified. In some embodiments, the N-terminus of the peptides or polypeptides described herein is acetylated (Ac=H₃C—CO—HN). In some embodiments, both the C-terminus and N-terminus of the peptides or polypeptides disclosed herein are modified. In some embodiments, the peptides and polypeptides disclosed herein have an amidated C-terminus and an acetylated N-terminus.

Conservative amino acid substitutions are those substitutions that, when made, least interfere with the properties of the original protein, that is, the structure and especially the function of the protein is conserved and not significantly changed by such substitutions. Examples of conservative substitutions are shown in the following table.

| Original Residue | Conservative Substitutions |
|---|---|
| Ala (A) | Ser |
| Arg (R) | Lys |
| Asn (N) | Gln, His |
| Asp (D) | Glu |
| Cys (C) | Ser |
| Gln (Q) | Asn |
| Glu (E) | Asp |
| His (H) | Asn; Gln |
| Ile (I) | Leu, Val |
| Leu (L) | Ile; Val |
| Lys (K) | Arg; Gln; Glu |
| Met (M) | Leu; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamine or aspartic acid; or (d) a residue having a bulky side chain, for example, phenylalanine, is substituted for (or by) one not having a side chain, for example, glycine.

Pharmaceutically acceptable carriers: The pharmaceutically acceptable carriers of use are conventional. Remington's Pharmaceutical Sciences, by E.W. Martin, Mack Publishing Co., Easton, Pa., 15th Edition, 1975, describes compositions and formulations suitable for pharmaceutical delivery of the compositions disclosed herein. In general, the nature of the carrier will depend on the particular mode of administration being employed. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Porphyrin: An organic compound containing four pyrrole rings, functioning as a metal-binding cofactor in hemoglobin, chlorophyll and certain enzymes.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques. The term recombinant includes nucleic acids and proteins that have been altered by addition, substitution, or deletion of a portion of a natural nucleic acid molecule or protein.

Sequence identity/similarity: The identity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Subject: Living multi-cellular organisms, including vertebrate organisms, a category that includes both human and non-human mammals.

Synthetic: Produced by artificial means in a laboratory, for example a synthetic polypeptide can be chemically synthesized in a laboratory.

Therapeutically effective amount: A quantity of compound or composition, for instance, a synthetic heme-containing molecule, sufficient to achieve a desired effect in a subject being treated. For instance, this can be the amount necessary to scavenge carbon monoxide in the blood or tissues, reduce the level of HbCO in the blood, and/or reduce one or more signs or symptoms associated with carbon monoxide poisoning, cyanide poisoning or H₂S poisoning.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Embodiments

A need exists for an effective, rapid and readily available therapy to treat carboxyhemoglobinemia, cyanide poisoning and hydrogen sulfide poisoning. It is disclosed herein that synthetic heme-containing molecules, bound to either two separate peptides or a single contiguous peptide, are capable of binding CO with high affinity and displacing CO from hemoglobin, thereby acting as CO scavengers.

A. Synthetic Heme-Containing Molecules

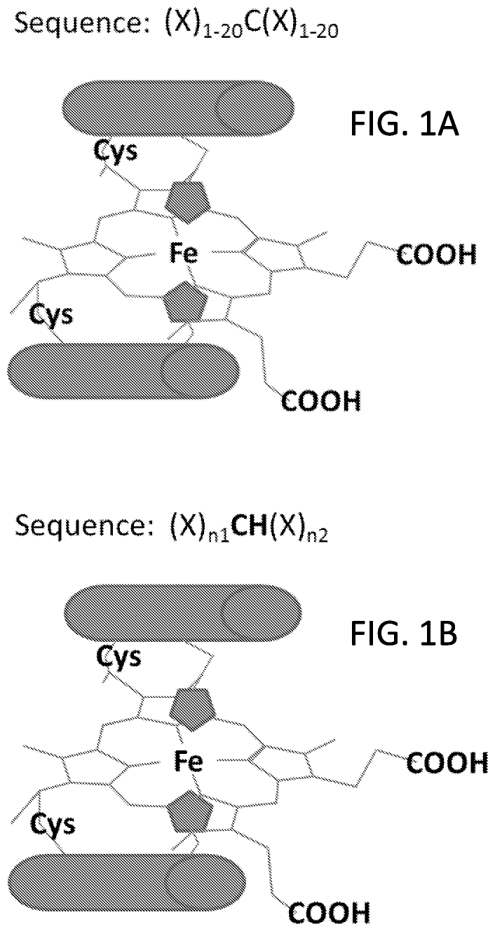

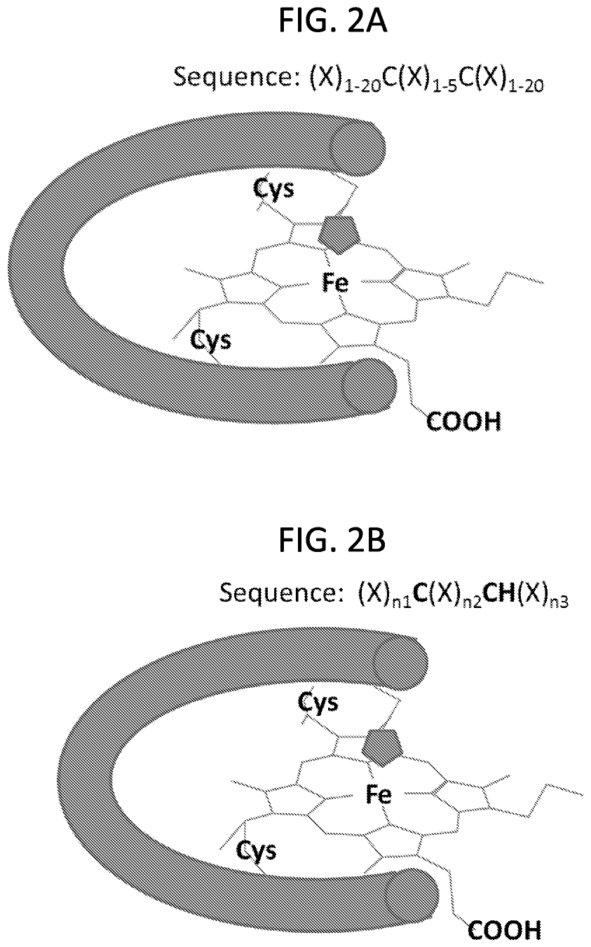

Described herein are synthetic heme-containing molecules. In some embodiments, the synthetic heme-containing molecules include a heme group bound to two non-contiguous peptides each having an amino acid sequence of the formula (X)₁₋₂₀C(X)₁₋₂₀ where each X is independently any natural or non-canonical amino acid, wherein C represents a cysteine residue and a cysteine residue of each peptide is bound to the heme group (see FIG. 1). In some embodiments, the two non-contiguous peptides each have an amino acid sequence of the formula (X)₁₋₁₅C(X)₁₋₁₅, (X)₁₋₁₀C(X)₁₋₁₀, or (X)₁₋₅C(X)₁₋₅. In other embodiments, the synthetic heme-containing molecules include a heme group bound to a single contiguous peptide having the formula (X)₁₋₂₀C(X)₁₋₅C(X)₁₋₂₀ (SEQ ID NO: 3), where each X is independently any natural or non-canonical amino acid, wherein C represents a cysteine residue and two cysteine residues of the peptide are bound to the heme group (see FIG. 2). In some embodiments, the single contiguous peptide has an amino acid sequence of the formula (X)₁₋₁₅C(X)₁₋₅C(X)₁₋₁₅, (X)₁₋₁₀C(X)₁₋₅C(X)₁₋₁₀, (X)₁₋₅C(X)₁₋₅C(X)₁₋₅, (X)₁₋₁₅C(X)₂₋₃C(X)₁₋₁₅, (X)₁₋₁₀C(X)₂₋₃C(X)₁₋₁₀ or (X)₁₋₅C(X)₂₋₃C(X)₁₋₅. In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments, the synthetic heme-containing molecule includes a heme group bound to two non-contiguous peptides, wherein the two non-contiguous peptides each have an amino acid sequence of the formula (X)₁₋₂₀CH(X)₁₋₁₉ (SEQ ID NO: 1) where each X is independently any natural or non-canonical amino acid, C represents a cysteine residue and H represents a histidine residue. In particular embodiments, the two non-contiguous peptides each have an amino acid sequence of the formula (X)₁₋₁₅CH(X)₁₋₁₄, (X)₁₋₁₀CH(X)₁₋₉, or (X)₁₋₅CH(X)₁₋₄. In one embodiment, the amino acid sequence of at least one of the two non-contiguous peptides comprises or consists of GCHGGR (SEQ ID NO: 2). In another non-limiting example, the amino acid sequence of both of the non-contiguous peptides comprises or consists of GCHGGR (SEQ ID NO: 2). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In other embodiments, the synthetic heme-containing molecule includes a heme group bound to a single contiguous peptide, wherein the peptide has an amino acid sequence of the formula (X)₁₋₂₀C(X)₁₋₂₀CH(X)₁₋₁₉ (SEQ ID NO: 4) where each X is independently any natural or non-canonical amino acid, C represents a cysteine residue and H represents a histidine residue. In particular embodiments, the peptide has an amino acid sequence of the formula (X)₁₋₁₅C(X)₁₋₅CH(X)₁₋₁₄, (X)₁₋₁₀C(X)₁₋₅CH(X)₁₋₉, (X)₁₋₅C(X)₁₋₅CH(X)₁₋₄, (X)₁₋₁₅C(X)₂₋₃CH(X)₁₋₁₄, (X)₁₋₁₀C(X)₂₋₃CH(X)₁₋₉ or (X)₁₋₅C(X)₂₋₃CH(X)₁₄. In one non-limiting example, the amino acid sequence of the peptide comprises or consists of QWGCGGCHG (SEQ ID NO: 5). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments, the synthetic heme-containing molecule includes a heme group bound to a single contiguous peptide, wherein the peptide has an amino acid sequence of the formula VQXCAQCX₁TVE (SEQ ID NO: 6) wherein X and X₁ are each independently any natural or non-canonical amino acid. In some embodiments, the synthetic heme-containing molecule includes a heme group bound to a single contiguous peptide, wherein the peptide has an amino acid sequence of the formula VQKCAQCHTVE (SEQ ID NO: 7). In some embodiments, the synthetic heme-containing molecule includes a heme group bound to a single contiguous peptide, wherein the peptide has an amino acid sequence of the formula VQECAQCHTVE (SEQ ID NO: 8). In some embodiments, the synthetic heme-containing molecule includes a heme group bound to a single contiguous peptide, wherein the peptide has an amino acid sequence of the formula VQKCAQCMTVE (SEQ ID NO: 9). In some embodiments, the synthetic heme-containing molecule includes a heme group bound to a single contiguous peptide, wherein the peptide has an amino acid sequence of the formula VQHCAQCHTVE (SEQ ID NO: 10). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments, the synthetic heme-containing molecule includes a heme group bound to two non-contiguous peptides, wherein the two non-contiguous peptides each have an amino acid sequence of the formula RCHGGR (SEQ ID NO: 11). In some embodiments, the synthetic heme-containing molecule includes a heme group bound to two non-contiguous peptides, wherein the two non-contiguous peptides each have an amino acid sequence of the formula GCHGGD (SEQ ID NO: 12). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH═CH₂) group. In some embodiments, the heme group is heme A, heme B, heme C, heme D, heme O, heme I, heme m, or heme S.

In some embodiments, the synthetic heme-containing molecule includes a heme group bound to a single contiguous peptide, wherein the peptide has an amino acid sequence of the formula QHGCGGCHG (SEQ ID NO: 13). In some embodiments, the synthetic heme-containing molecule includes a heme group bound to a single contiguous peptide, wherein the peptide has an amino acid sequence of the formula QHGCGGCGHG (SEQ ID NO: 14). In some embodiments, the synthetic heme-containing molecule includes a heme group bound to a single contiguous peptide, wherein the peptide has an amino acid sequence of the formula QHGGCGGCHG (SEQ ID NO: 15). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments, the peptide(s) of the synthetic heme-containing molecule includes at least one modification, such as an N-terminal modification, a C-terminal modification or both an N-terminal modification and a C-terminal modification. N-terminal modifications include, but are not limited to, acetylation, formylation, propionylation, pyroglutamate formation, myristoylation, palmitylation, S-palmitoylation, mono-methylation, di-methylation, or tri-methylation. In particular examples, the N-terminal modification is acetylation. C-terminal modifications include, but are not limited to, amidation and methylation. In non-limiting examples, the N-terminus of the peptide is acetylated and the C-terminal is amidated.

In some embodiments, direct modification of the porphyrin macrocycle or changing the metal center is used to tune the electronic properties of the synthetic heme-containing molecule in order to augment both oxygen and CO affinity of the molecule. The present disclosure contemplates modifications that minimize oxygen affinity (and autoxidation) while maintaining CO affinity of the synthetic heme-containing molecules. In some embodiments, modified heme groups include tetrapyrrole macrocycles such as corrins (e.g., in Vitamin B₁₂) and the related fully aromatic corrole. These macrocyclic ligands are similar to porphyrins, except they both have a limited physical metal-binding cavity due to a direct C₁ to C₁₉ bipyrrole linkage (i.e., the loss of a methylene linker compared to porphyrin) and have considerably altered electronic properties as corrins are monoanionic and corroles are trianionic. In some embodiments, the heme groups are a nontraditional dianionic porphyrin. The use of non-traditional dianionic porphyrins allows for more incremental changes to the electronics of the heme group and synthetic heme-containing molecule. Mammalian globin proteins and microperoxidases typically use a protoporphyrin IX macrocyclic structure. For example, chlorin e6, a porphyrin derivative native to chlorophyll where one pyrrole is only partially electronically saturated and with three carboxylic moieties, is more electronically deficient than the protoporphyrin IX equivalent, meaning, when chelating iron, the affinity for oxygen decreases (Sreenilayam et al., ACS Catal 7(11): 7629-7633, 2017). Chlorin e6 still contains a singular vinyl group; formation of one thioether bond still viable, although “sandwich”-type proteins may not be possible; thus peptides for these molecules may contain only one cysteine. In some embodiments, changing the iron to another redox active metal such as another Group VIII metal like ruthenium (Ru) or a group IX metal such as cobalt (Co), may directly modify the electronics of the CO binding moiety of the synthetic heme-containing molecule. Moreover, Co(III) porphyrins (which do not interact with oxygen) have been indicated to bind CO (Brown et al., J. Am. Chem. Soc. 93 (7), 1790-1791, 1971; Schmidt et al., J. Am. Chem. Soc. 118 (12), 2954-2961, 1996). In some embodiments, the heme group is a microperoxidase where the iron is exchanged for cobalt. In some embodiments, cobalt is paired with the trianionic corrole inside a heme group, thereby stabilizing the neutral, cobalt(III) complex which binds CO (Guilard et al., Inorg. Chem. 40 (19), 4845-4855, 2001; Barbe et al., Dalton Trans. No. 8, 1208-1214, 2004). In some embodiments, these modified heme groups rely on distal histidine binding to peptides, binding to vinyl groups on a corrin macrocycle or any combination thereof.

B. Methods of Treating Carboxyhemoglobinemia

Further described herein are methods of removing carbon monoxide from hemoglobin in blood or animal tissue. In some embodiments, the method includes contacting the blood or animal tissue with a synthetic heme-containing molecule disclosed herein. In some embodiments, the method is an in vitro method. In other embodiments, the method is an in vivo method, wherein contacting the blood or animal tissue with the synthetic heme-containing molecule comprises administering a therapeutically effective amount of the synthetic heme-containing molecule to a subject.

Also described herein is a method of treating carboxyhemoglobinemia in a subject. In some embodiments, the method includes administering to the subject a synthetic heme-containing molecule disclosed herein.

In some embodiments of these methods, the synthetic heme-containing molecules include a heme group bound to two non-contiguous peptides each having an amino acid sequence of the formula (X)₁₋₂₀C(X)₁₋₂₀ where X is any natural or non-canonical amino acid, wherein C represents a cysteine residue and a cysteine residue of each peptide is bound to the heme group (FIG. 1). In some embodiments, the two non-contiguous peptides each have an amino acid sequence of the formula (X)₁₋₁₅C(X)₁₋₁₅, (X)₁₋₁₀C(X)₁₋₁₀, or (X)₁₋₅C(X)₁₋₅. In other embodiments, the synthetic heme-containing molecules include a heme group bound to a single contiguous peptide having the formula (X)₁₋₂₀C(X)₁₋₂₀C(X)₁₋₂₀ (SEQ ID NO: 3) where X is any natural or non-canonical amino acid, wherein C represents a cysteine residue and two cysteine residues of the peptide are bound to the heme group (FIG. 2). In some embodiments, the single contiguous peptide has an amino acid sequence of the formula (X)₁₋₁₅C(X)₁₋₅C(X)₁₋₁₅, (X)₁₋₁₀C(X)₁₋₅C(X)₁₋₁₀, (X)₁₋₅C(X)₁₋₅C(X)₁₋₅, (X)₁₋₁₅C(X)₂₋₃C(X)₁₋₁₅, (X)₁₋₁₀C(X)₂₋₃C(X)₁₋₁₀ or (X)₁₋₅C(X)₂₋₃C(X)₁₋₅. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH═CH₂) group. In some embodiments, the heme group is heme A, heme B, heme C, heme D, heme O, heme I, heme m, or heme S.

In some embodiments of these methods, the synthetic heme-containing molecule includes a heme group bound to two non-contiguous peptides, wherein the two non-contiguous peptides each have an amino acid sequence of the formula (X)₁₋₂₀CH(X)₁₋₁₉ (SEQ ID NO: 1) where X is any natural or non-canonical amino acid, C represents a cysteine residue and H represents a histidine residue. In particular embodiments, the two non-contiguous peptides each have an amino acid sequence of the formula (X)₁₋₁₅CH(X)₁₋₁₄, (X)₁₋₁₀CH(X)₁₋₉, or (X)₁₋₅CH(X)₁₋₄. In one non-limiting example, the amino acid sequence of at least one of the two non-contiguous peptides comprises or consists of GCHGGR (SEQ ID NO: 2). In another non-limiting example, the amino acid sequence of both of the non-contiguous peptides comprises or consists of GCHGGR (SEQ ID NO: 2). In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH═CH₂) group. In some embodiments, the heme group is heme A, heme B, heme C, heme D, heme O, heme I, heme m, or heme S.

In other embodiments, the synthetic heme-containing molecule includes a heme group bound to a single contiguous peptide, wherein the peptide has an amino acid sequence of the formula (X)₁₋₂₀C(X)₁₋₂₀CH(X)₁₋₁₉ (SEQ ID NO: 4) where X is any natural or non-canonical amino acid, C represents a cysteine residue and H represents a histidine residue. In particular embodiments, the peptide has an amino acid sequence of the formula (X)₁₋₁₅C(X)₁₋₅CH(X)₁₋₁₄, (X)₁₋₁₀C(X)₁₋₅CH(X)₁₋₉, (X)₁₋₅C(X)₁₋₅CH(X)₁₋₄, (X)₁₋₁₅C(X)₂₋₃CH(X)₁₋₁₄, (X)₁₋₁₀C(X)₂₋₃CH(X)₁₋₉ or (X)₁₋₅C(X)₂₋₃CH(X)₁₋₄. In one non-limiting example, the amino acid sequence of the peptide comprises or consists of QWGCGGCHG (SEQ ID NO: 5). In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments of these methods, the synthetic heme-containing molecule includes a heme group bound to a single contiguous peptide, wherein the peptide has an amino acid sequence of the formula VQXCAQCX₁TVE (SEQ ID NO: 6) wherein X and X₁ are each independently any natural or non-canonical amino acid. In some embodiments, the synthetic heme-containing molecule includes a heme group bound to a single contiguous peptide, wherein the peptide has an amino acid sequence of the formula VQKCAQCHTVE (SEQ ID NO: 7). In some embodiments, the synthetic heme-containing molecule includes a heme group bound to a single contiguous peptide, wherein the peptide has an amino acid sequence of the formula VQECAQCHTVE (SEQ ID NO: 8). In some embodiments, the synthetic heme-containing molecule includes a heme group bound to a single contiguous peptide, wherein the peptide has an amino acid sequence of the formula VQKCAQCMTVE (SEQ ID NO: 9). In some embodiments, the synthetic heme-containing molecule includes a heme group bound to a single contiguous peptide, wherein the peptide has an amino acid sequence of the formula VQHCAQCHTVE (SEQ ID NO: 10). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments, the synthetic heme-containing molecule includes a heme group bound to two non-contiguous peptides, wherein the two non-contiguous peptides each have an amino acid sequence of the formula RCHGGR (SEQ ID NO: 11). In some embodiments, the synthetic heme-containing molecule includes a heme group bound to two non-contiguous peptides, wherein the two non-contiguous peptides each have an amino acid sequence of the formula GCHGGD (SEQ ID NO: 12). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH═CH₂) group. In some embodiments, the heme group is heme A, heme B, heme C, heme D, heme O, heme I, heme m, or heme S.

In some embodiments, the synthetic heme-containing molecule includes a heme group bound to a single contiguous peptide, wherein the peptide has an amino acid sequence of the formula QHGCGGCHG (SEQ ID NO: 13). In some embodiments, the synthetic heme-containing molecule includes a heme group bound to a single contiguous peptide, wherein the peptide has an amino acid sequence of the formula QHGCGGCGHG (SEQ ID NO: 14). In some embodiments, the synthetic heme-containing molecule includes a heme group bound to a single contiguous peptide, wherein the peptide has an amino acid sequence of the formula QHGGCGGCHG (SEQ ID NO: 15). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments of these methods, the peptide(s) of the synthetic heme-containing molecule includes at least one modification, such as an N-terminal modification, a C-terminal modification or both an N-terminal modification and a C-terminal modification. N-terminal modifications include, but are not limited to, acetylation, formylation, propionylation, pyroglutamate formation, myristoylation, palmitylation, S-palmitoylation, mono-methylation, di-methylation, or tri-methylation. In particular examples, the N-terminal modification is acetylation. C-terminal modifications include, but are not limited to, amidation and methylation. In non-limiting examples, the N-terminus of the peptide is acetylated and the C-terminal is amidated.

In some embodiments, the disclosed methods further include selecting a subject with carboxyhemoglobinemia prior to administering the synthetic heme-containing molecule to the subject. In some embodiments, the methods further include testing the level of carboxyhemoglobin in a subject, such as to enable selection of a subject with carboxyhemoglobinemia. In some embodiments, the subject has at least 5%, at least 10%, at least 15%, at least 20%, at least 30%, at least 40% or at least 50% carboxyhemoglobin in their blood prior to treatment. Methods for measuring HbCO, such as by spectrophotometric or chromatographic methods, are well known in the art (see, e.g., U.S. Application Publication No. 2003/0202170; Rodkey et al., Clin Chem 25(8):1388-1393, 1979; Barker et al., Anesthesiology 105(5):892-897, 2006).

In some embodiments, the synthetic heme-containing molecule is administered by intravenous infusion.

In some embodiments, the synthetic heme-containing molecule is administered to a subject at a dose of about 0.1 gram to about 300 grams, such as about 1 gram to about 200 grams, 10 grams to about 100 grams, about 10 grams to about 50 grams, about 30 grams to about 300 grams, or about 30 grams to about 150 grams. In particular embodiments, the synthetic heme-containing molecule is administered to a subject at a dose of about 0.1, about 0.5, about 1, about 10, about 20, about 30, about 40, about 50, about 60, about 70, about 80, about 90, about 100, about 125, about 150, about 175, about 200, about 225, about 250 or about 300 grams.

The synthetic heme-containing molecule can be administered to a subject in a single dose, or in multiple doses as needed, to reduce HbCO to a non-toxic level.

In some embodiments, the dose administered to the subject is the amount of synthetic heme-containing molecule required to reduce HbCO by at least 1%, at least 2%, at least 3%, at least 4%, at least 5%, at least 10%, at least 15%, at least 20%, at least 25%, at least 30%, at least 40%, at least 50%, at least 60%, at least 70%, at least 80% or at least 90% (compared to the level of HbCO before treatment) in blood and/or tissue of the subject.

C. Methods of Treating Cyanide Poisoning

Cyanide is known to inhibit mitochondrial respiration, in a similar manner to CO-mediated inhibition of mitochondrial respiration by binding to the heme a3 center in cytochrome c oxidase. Although it partially binds the reduced form, cyanide binds strongest to the oxidized state of cytochrome c oxidase (complex IV of the electron transport chain) (Leavesley et al., Toxicol Sci 101(1):101-111, 2008). Similar to a small heme-containing molecule's ability to scavenge CO, these molecules are able to scavenge cyanide. Thus, the use of synthetic heme-containing molecules for removing cyanide from cyano-hemoglobin located inside red blood cells, as well as other heme containing proteins in the body (such as cytochrome c oxidase), is contemplated herein.

Described herein are methods of removing cyanide from a heme protein (such as hemoglobin or cytochrome c oxidase) in blood or animal tissue. In some embodiments, the method includes contacting the blood or animal tissue with a synthetic heme-containing molecule disclosed herein. In some embodiments, the method is an in vitro method. In other embodiments, the method is an in vivo method, wherein contacting the blood or animal tissue with the synthetic heme-containing molecule comprises administering a therapeutically effective amount of the synthetic heme-containing molecule to a subject.

Also described is a method of treating cyanide poisoning in a subject. In some embodiments, the method includes administering to the subject a synthetic heme-containing molecule disclosed herein.

In some embodiments of these methods, the synthetic heme-containing molecules include a heme group bound to two non-contiguous peptides each having an amino acid sequence of the formula (X)₁₋₂₀C(X)₁₋₂₀ where X is any natural or non-canonical amino acid, wherein C represents a cysteine residue and a cysteine residue of each peptide is bound to the heme group (FIG. 1). In some embodiments, the two non-contiguous peptides each have an amino acid sequence of the formula (X)₁₋₁₅C(X)₁₋₁₅, (X)₁₋₁₀C(X)₁₋₁₀, or (X)₁₋₅C(X)₁₋₅. In other embodiments, the synthetic heme-containing molecules include a heme group bound to a single contiguous peptide having the formula (X)₁₋₂₀C(X)₁₋₂₀C(X)₁₋₂₀ (SEQ ID NO: 3) where each X is independently any natural or non-canonical amino acid, wherein C represents a cysteine residue and two cysteine residues of the peptide are bound to the heme group (FIG. 2). In some embodiments, the single contiguous peptide has an amino acid sequence of the formula (X)₁₋₁₅C(X)₁₋₅C(X)₁₋₁₅, (X)₁₋₁₀C(X)₁₋₅C(X)₁₋₁₀, (X)₁₋₅C(X)₁₋₅C(X)₁₋₅, (X)₁₋₁₅C(X)₂₋₃C(X)₁₋₁₅, (X)₁₋₁₀C(X)₂₋₃C(X)₁₋₁₀ or (X)₁₋₅C(X)₂₋₃C(X)₁₋₅. In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments of these methods, the synthetic heme-containing molecule includes a heme group bound to two non-contiguous peptides, wherein the two non-contiguous peptides each have an amino acid sequence of the formula (X)₁₋₂₀CH(X)₁₋₁₉ (SEQ ID NO: 1) where each X is independently any natural or non-canonical amino acid, C represents a cysteine residue, and H represents a histidine residue. In particular embodiments, the two non-contiguous peptides each have an amino acid sequence of the formula (X)₁₋₁₅CH(X)₁₋₁₄, (X)₁₋₁₀CH(X)₁₋₉, or (X)₁₋₅CH(X)₁₋₄. In one non-limiting example, the amino acid sequence of at least one of the two non-contiguous peptides comprises or consists of GCHGGR (SEQ ID NO: 2). In another non-limiting example, the amino acid sequence of both of the non-contiguous peptides comprises or consists of GCHGGR (SEQ ID NO: 2). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH═CH₂) group. In some embodiments, the heme group is heme A, heme B, heme C, heme D, heme O, heme I, heme m, or heme S.

In other embodiments, the synthetic heme-containing molecule includes a heme group bound to a single contiguous peptide, wherein the peptide has an amino acid sequence of the formula (X)₁₋₂₀C(X)₁₋₂₀CH(X)₁₋₁₉ (SEQ ID NO: 4) where each X is independently any natural or non-canonical amino acid, C represents a cysteine residue, and H represents a histidine residue. In particular embodiments, the peptide has an amino acid sequence of the formula (X)₁₋₁₅C(X)₁₋₅CH(X)₁₋₁₄, (X)₁₋₁₀C(X)₁₋₅CH(X)₁₋₉, (X)₁₋₅C(X)₁₋₅CH(X)₁₋₄, (X)₁₋₁₅C(X)₂₋₃CH(X)₁₋₁₄, (X)₁₋₁₀C(X)₂₋₃CH(X)₁₋₉ or (X)₁₋₅C(X)₂₋₃CH(X)₁₋₄. In one non-limiting example, the amino acid sequence of the peptide comprises or consists of QWGCGGCHG (SEQ ID NO: 5). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments of these methods, the synthetic heme-containing molecule includes a heme group bound to a single contiguous peptide, wherein the peptide has an amino acid sequence of the formula VQXCAQCX₁TVE (SEQ ID NO: 6) wherein X and X₁ are each independently any natural or non-canonical amino acid. In some embodiments, the synthetic heme-containing molecule includes a heme group bound to a single contiguous peptide, wherein the peptide has an amino acid sequence of the formula VQKCAQCHTVE (SEQ ID NO: 7). In some embodiments, the synthetic heme-containing molecule includes a heme group bound to a single contiguous peptide, wherein the peptide has an amino acid sequence of the formula VQECAQCHTVE (SEQ ID NO: 8). In some embodiments, the synthetic heme-containing molecule includes a heme group bound to a single contiguous peptide, wherein the peptide has an amino acid sequence of the formula VQKCAQCMTVE (SEQ ID NO: 9). In some embodiments, the synthetic heme-containing molecule includes a heme group bound to a single contiguous peptide, wherein the peptide has an amino acid sequence of the formula VQHCAQCHTVE (SEQ ID NO: 10). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments, the synthetic heme-containing molecule includes a heme group bound to two non-contiguous peptides, wherein the two non-contiguous peptides each have an amino acid sequence of the formula RCHGGR (SEQ ID NO: 11). In some embodiments, the synthetic heme-containing molecule includes a heme group bound to two non-contiguous peptides, wherein the two non-contiguous peptides each have an amino acid sequence of the formula GCHGGD (SEQ ID NO: 12). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH═CH₂) group. In some embodiments, the heme group is heme A, heme B, heme C, heme D, heme O, heme I, heme m, or heme S.

In some embodiments, the synthetic heme-containing molecule includes a heme group bound to a single contiguous peptide, wherein the peptide has an amino acid sequence of the formula QHGCGGCHG (SEQ ID NO: 13). In some embodiments, the synthetic heme-containing molecule includes a heme group bound to a single contiguous peptide, wherein the peptide has an amino acid sequence of the formula QHGCGGCGHG (SEQ ID NO: 14). In some embodiments, the synthetic heme-containing molecule includes a heme group bound to a single contiguous peptide, wherein the peptide has an amino acid sequence of the formula QHGGCGGCHG (SEQ ID NO: 15). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments of these methods, the peptide(s) of the synthetic heme-containing molecule includes at least one modification, such as an N-terminal modification, a C-terminal modification or both an N-terminal modification and a C-terminal modification. N-terminal modifications include, but are not limited to, acetylation, formylation, propionylation, pyroglutamate formation, myristoylation, palmitylation, S-palmitoylation, mono-methylation, di-methylation, or tri-methylation. In particular examples, the N-terminal modification is acetylation. C-terminal modifications include, but are not limited to, amidation and methylation. In non-limiting examples, the N-terminus of the peptide is acetylated and the C-terminal is amidated.

In some embodiments, the disclosed methods further include selecting a subject with cyanide poisoning prior to administering the synthetic heme-containing molecule to the subject. In some embodiments, the methods further include testing the level of cyanide in a subject, such as to enable selection of a subject with cyanide poisoning. In some embodiments, the subject has at least 0.5-1.0 μg/mL red blood cell cyanide concentration, at least 0.5 to 1 mg/L (12 to 23 μmol/L) blood cyanide concentrations in their blood prior to treatment or positive Cyantesmo test strips (colorimetric strips for presence of cyanide) from their blood prior to treatment.

In some embodiments, the synthetic heme-containing molecule is administered by intravenous infusion.

In some embodiments, the synthetic heme-containing molecule is administered to a subject at a dose of about 0.1 gram to about 300 grams, such as about 1 gram to about 200 grams, about 10 grams to about 100 grams, about 10 grams to about 50 grams, about 30 grams to about 300 grams, or about 30 grams to about 150 grams. In particular embodiments, the synthetic heme-containing molecule is administered to a subject at a dose of about 0.1, about 0.5, about 1, about 10, about 20, about 30, about 40, about 50, about 60, about 70, about 80, about 90, about 100, about 125, about 150, about 175, about 200, about 225, about 250 or about 300 grams.

The synthetic heme-containing molecule can be administered to a subject in a single dose, or in multiple doses as needed, to reduce cyano-hemoglobin to a non-toxic level.

In some embodiments, the dose administered to the subject is the amount of synthetic heme-containing molecule required to reduce blood cyanide levels. In some embodiments, the subject will have at least 0.5-1.0 μg/mL red blood cell cyanide concentration, at least 0.5 to 1 mg/L (12 to 23 μmol/L) blood cyanide concentrations in their blood prior to treatment or positive Cyantesmo test strips (colorimetric strips for presence of cyanide) from their blood prior to treatment. In some embodiments, the dose administered to the subject is the amount of synthetic heme-containing molecule required to reduce blood cyanide levels by at least 1%, at least 2%, at least 3%, at least 4%, at least 5%, at least 10%, at least 15%, at least 20%, at least 25%, at least 30%, at least 40%, at least 50%, at least 60%, at least 70%, at least 80% or at least 90% (compared to the level of cyanide before treatment) in blood of the subject.

D. Methods of Treating Hydrogen Sulfide (H₂S) Poisoning

Hydrogen sulfide is known to inhibit mitochondrial respiration, in a similar manner to CO-mediated inhibition of mitochondrial respiration. H₂S binds strongest to the reduced form of cytochrome c oxidase (complex IV of the electron transport chain) (Nicholls et al., Biochem Soc Trans 41(5):1312-1316, 2013). Similar to a small heme-containing molecule's ability to scavenge CO, these molecules are also able to scavenge H₂S. Thus, the use of synthetic heme-containing molecules for removing H₂S from hemoglobin located inside red blood cells, as well as other heme containing proteins in the body (such as cytochrome c oxidase), is contemplated herein.

Described herein are methods of removing H₂S from a heme protein (such as hemoglobin or cytochrome c oxidase) in blood or animal tissue. In some embodiments, the method includes contacting the blood or animal tissue with a synthetic heme-containing molecule disclosed herein. In some embodiments, the method is an in vitro method. In other embodiments, the method is an in vivo method, wherein contacting the blood or animal tissue with the synthetic heme-containing molecule comprises administering a therapeutically effective amount of the synthetic heme-containing molecule to a subject.

Also described is a method of treating H₂S poisoning in a subject. In some embodiments, the method includes administering to the subject a synthetic heme-containing molecule disclosed herein.

In some embodiments of these methods, the synthetic heme-containing molecules include a heme group bound to two non-contiguous peptides each having an amino acid sequence of the formula (X)₁₋₂₀C(X)₁₋₂₀ where X is any natural or non-canonical amino acid, wherein C represents a cysteine residue and a cysteine residue of each peptide is bound to the heme group (FIG. 1). In some embodiments, the two non-contiguous peptides each have an amino acid sequence of the formula (X)₁₋₁₅C(X)₁₋₁₅, (X)₁₋₁₀C(X)₁₋₁₀, or (X)₁₋₅C(X)₁₋₅. In other embodiments, the synthetic heme-containing molecules include a heme group bound to a single contiguous peptide having the formula (X)₁₋₂₀C(X)₁₋₂₀C(X)₁₋₂₀ (SEQ ID NO: 3) where X is any natural or non-canonical amino acid, wherein two cysteine residues of the peptide are bound to the heme group (FIG. 2). In some embodiments, the single contiguous peptide has an amino acid sequence of the formula (X)₁₋₁₅C(X)₁₋₅-C(X)₁₋₁₅, (X)₁₋₁₀C(X)₁₋₅C(X)₁₋₁₀, (X)₁₋₅C(X)₁₋₅C(X)₁₋₅, (X)₁₋₁₅C(X)₂₋₃C(X)₁₋₁₅, (X)₁₋₁₀C(X)₂₋₃C(X)₁₋₁₀ or (X)₁₋₅C(X)₂₋₃C(X)₁₋₅.

In some embodiments of these methods, the synthetic heme-containing molecule includes a heme group bound to two non-contiguous peptides, wherein the two non-contiguous peptides each have an amino acid sequence of the formula (X)₁₋₂₀CH(X)₁₋₁₉ (SEQ ID NO: 1) where X is any natural or non-canonical amino acid, C represents a cysteine residue, and H represents a histidine residue. In particular embodiments, the two non-contiguous peptides each have an amino acid sequence of the formula (X)₁₋₁₅CH(X)₁₋₁₄, (X)₁₋₁₀CH(X)₁₋₉, or (X)₁₋₅CH(X)₁₋₄. In one non-limiting example, the amino acid sequence of at least one of the two non-contiguous peptides comprises or consists of GCHGGR (SEQ ID NO: 2). In another non-limiting example, the amino acid sequence of both of the non-contiguous peptides comprises or consists of GCHGGR (SEQ ID NO: 2).

In other embodiments, the synthetic heme-containing molecule includes a heme group bound to a single contiguous peptide, wherein the peptide has an amino acid sequence of the formula (X)₁₋₂₀C(X)₁₋₂₀CH(X)₁₋₁₉ (SEQ ID NO: 4) where X is any natural or non-canonical amino acid, C represents a cysteine residue, and H represents a histidine residue. In particular embodiments, the peptide has an amino acid sequence of the formula (X)₁₋₁₅C(X)₁₋₅CH(X)₁₋₁₄, (X)₁₋₁₀C(X)₁₋₅CH(X)₁₋₉, (X)₁₋₅C(X)₁₋₅CH(X)₁₋₄, (X)₁₋₁₅IC(X)₂₋₃CH(X)₁₋₁₄, (X)₁₋₁₀C(X)₂₋₃CH(X)₁₋₉ or (X)₁₋₅ C(X)₂₋₃CH(X)₁₋₄. In one non-limiting example, the amino acid sequence of the peptide comprises or consists of QWGCGGCHG (SEQ ID NO: 5). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments of these methods, the synthetic heme-containing molecule includes a heme group bound to a single contiguous peptide, wherein the peptide has an amino acid sequence of the formula VQXCAQCX₁TVE (SEQ ID NO: 6) wherein X and X₁ are each independently any natural or non-canonical amino acid. In some embodiments, the synthetic heme-containing molecule includes a heme group bound to a single contiguous peptide, wherein the peptide has an amino acid sequence of the formula VQKCAQCHTVE (SEQ ID NO: 7). In some embodiments, the synthetic heme-containing molecule includes a heme group bound to a single contiguous peptide, wherein the peptide has an amino acid sequence of the formula VQECAQCHTVE (SEQ ID NO: 8). In some embodiments, the synthetic heme-containing molecule includes a heme group bound to a single contiguous peptide, wherein the peptide has an amino acid sequence of the formula VQKCAQCMTVE (SEQ ID NO: 9). In some embodiments, the synthetic heme-containing molecule includes a heme group bound to a single contiguous peptide, wherein the peptide has an amino acid sequence of the formula VQHCAQCHTVE (SEQ ID NO: 10). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments, the synthetic heme-containing molecule includes a heme group bound to two non-contiguous peptides, wherein the two non-contiguous peptides each have an amino acid sequence of the formula RCHGGR (SEQ ID NO: 11). In some embodiments, the synthetic heme-containing molecule includes a heme group bound to two non-contiguous peptides, wherein the two non-contiguous peptides each have an amino acid sequence of the formula GCHGGD (SEQ ID NO: 12). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH═CH₂) group. In some embodiments, the heme group is heme A, heme B, heme C, heme D, heme O, heme I, heme m, or heme S.

In some embodiments, the synthetic heme-containing molecule includes a heme group bound to a single contiguous peptide, wherein the peptide has an amino acid sequence of the formula QHGCGGCHG (SEQ ID NO: 13). In some embodiments, the synthetic heme-containing molecule includes a heme group bound to a single contiguous peptide, wherein the peptide has an amino acid sequence of the formula QHGCGGCGHG (SEQ ID NO: 14). In some embodiments, the synthetic heme-containing molecule includes a heme group bound to a single contiguous peptide, wherein the peptide has an amino acid sequence of the formula QHGGCGGCHG (SEQ ID NO: 15). In some embodiments, the heme group is a microperoxidase. In some embodiments, the microperoxidase is synthetic microperoxidase 11. In some embodiments, the heme group is a metal porphyrin. In yet other embodiments, the heme group is a porphyrin with vinyl groups at carbons C3, C8, or both C3 and C8. In some embodiments the metal porphyrin is an iron porphyrin, or a cobalt porphyrin. In some embodiments, the iron porphyrin is ferriprotoporphyrin IX chloride (Hemin). In some embodiments, the cobalt porphyrin is protoporphyrin IX cobalt chloride. In some embodiments, the heme group is any natural or synthetic heme with a vinyl (—CH—CH₂) group. In some embodiments, the heme group is heme A, heme B, heme C, heme D, heme O, heme I, heme m, or heme S.

In some embodiments of these methods, the peptide(s) of the synthetic heme-containing molecule includes at least one modification, such as an N-terminal modification, a C-terminal modification or both an N-terminal modification and a C-terminal modification. N-terminal modifications include, but are not limited to, acetylation, formylation, propionylation, pyroglutamate formation, myristoylation, palmitylation, S-palmitoylation, mono-methylation, di-methylation, or tri-methylation. In particular examples, the N-terminal modification is acetylation. C-terminal modifications include, but are not limited to, amidation and methylation. In non-limiting examples, the N-terminus of the peptide is acetylated and the C-terminal is amidated.

In some embodiments, the disclosed methods further include selecting a subject with H₂S poisoning prior to administering the synthetic heme-containing molecule to the subject. In some embodiments, the methods further include testing the level of H₂S-hemoglobin in a subject, such as to enable selection of a subject with H₂S poisoning after known exposure to H₂S gas (as low as 2 parts per million). In some embodiments, the subject has at least 0.5%, at least 1%, at least 5%, at least 10%, at least 15%, at least 20%, at least 30%, at least 40% or at least 50% sulfhemoglobin in their blood or a narrowed venous-arterial PO₂ gradient prior to treatment.

In some embodiments, the synthetic heme-containing molecule is administered by intravenous infusion.

In some embodiments, the synthetic heme-containing molecule is administered to a subject at a dose of about 0.1 gram to about 300 grams, about 1 gram to about 200 grams, such as about 10 grams to about 100 grams, about 10 grams to about 50 grams, about 30 grams to about 300 grams, or about 30 grams to about 150 grams. In particular embodiments, the synthetic heme-containing molecule is administered to a subject at a dose of about 0.1, about 0.5, about 1, about 10, about 20, about 30, about 40, about 50, about 60, about 70, about 80, about 90, about 100, about 125, about 150, about 175, about 200, about 225, about 250 or about 300 grams.

The synthetic heme-containing molecule can be administered to a subject in a single dose, or in multiple doses as needed, to reduce H₂S-hemoglobin to a non-toxic level.

In some embodiments, the dose administered to the subject is the amount of synthetic heme-containing molecule required to reduce sulfhemoglobin by at least 0.5%, at least 1%, at least 2%, at least 3%, at least 4%, at least 5%, at least 10%, at least 15%, at least 20%, at least 25%, at least 30%, at least 40%, at least 50%, at least 60%, at least 70%, at least 80% or at least 90% (compared to the level of H₂S-hemoglobin before treatment) in blood and/or tissue of the subject.

The following examples are provided to illustrate certain particular features and/or embodiments. These examples should not be construed to limit the disclosure to the particular features or embodiments described.

### EXAMPLES

### Example 1: Synthetically Created Heme Molecules can be Reduced with Dithionite and Bind to Carbon Monoxide when Exposed to CO Gas

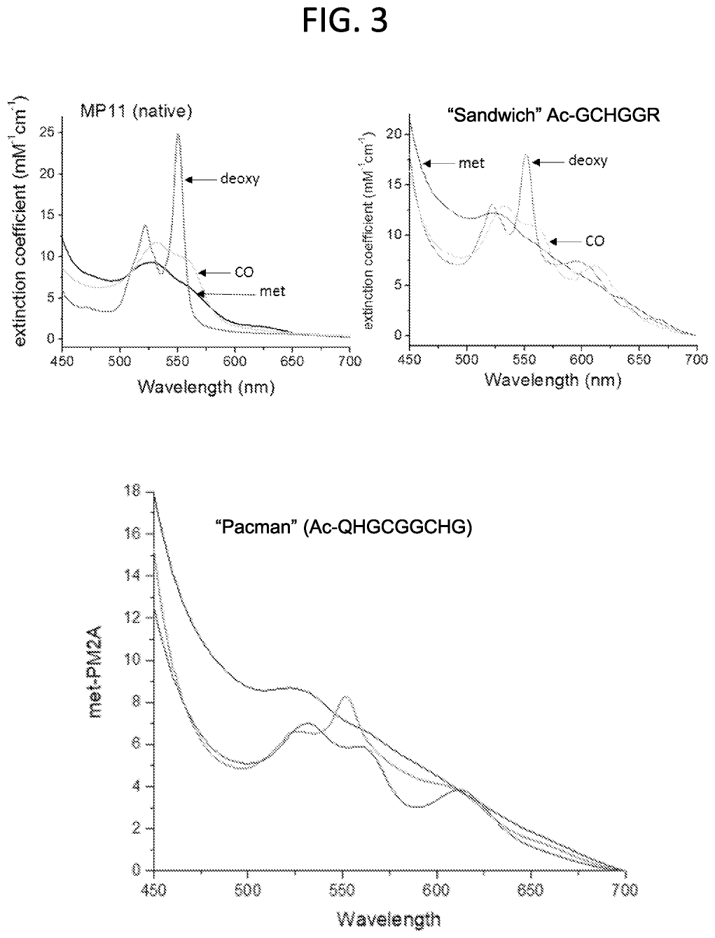

A heme molecule bound to two peptide chains Ac-GCHGGR (SEQ ID NO: 2) in a “sandwich” (see FIG. 1), when prepared in anaerobic conditions, changes absorbance spectra (measured by Cary Spec device) with the introduction of stoichiometric amounts of dithionite. When a CO saturated aqueous solution (phosphate buffered saline) is added, the absorbance spectra changes, indicating its CO binding ability (FIG. 3).

### Example 2: Synthetic Microperoxidase Analogue Production

Solid phase peptide synthesis (SPPS) of synthetic peptide analogues was carried out using FMOC chemistry and Oxyma/DIC activation on a CEM Liberty Blue microwave synthesizer using Wang resin as solid support. After completion of peptide chain assembly, the N-terminus of the peptide resins were acetylated with 50% acetic anhydride/pyridine for 1 hour at room temperature. Cleavage of the resulting acetylated peptides from the Wang resin was accomplished using TFA:Thionanisole:Anisole:Ethaneditiol (90:5:2:3) for 2 hours at room temperature and then precipitated with diethyl ether. Residual scavengers were extracted from the crude peptides by 3 rounds of diethyl ether washes followed by centrifugation steps. The resulting crude peptides were then purified by preparative C-18 reverse phase (RP)-HPLC (250×21.2 mm column) using a Waters Delta Prep 4000 chromatography system and standard water and acetonitrile/0.1% TFA gradient conditions followed by lyophilization. Analytical C-18 RP-HPLC (250×4.6 mm column) characterization on a Waters Alliance chromatography system followed by MALDI-TOF analysis using alpha-cyano-4-hydroxy-cinnamic acid (CHCA) matrix conditions was performed on an Applied Biosystems Voyager workstation to confirm the expected mass and purity of the final acetylated peptide products. Conjugation of synthetic acetylated peptide analogues to heme groups for production of either “PACMAN” or “Sandwich” type constructs was accomplished under mild aqueous conditions at room temperature using an adaptation of methods previously utilized to form c-type cytochrome protein variants (Daltrop et al., J Biol Chem 278(27):24308-24313, 2003). For production of the “PACMAN” type construct, 20 mg (2.25 mM total) ferriprotoporphyrin IX chloride (Hemin) (or alternatively protoporphyrin IX cobalt chloride) was activated and dissolved in 2 ml of 0.1 M NaOH (pH 13) at room temperature for several minutes and then slowly added dropwise to a 12 ml solution of 50 mM phosphate buffer at pH 7.0/ethanol (1:1)(v:v) containing 0.25 mM of the disulfide containing peptide along with 25 mM dithionite and 25 mM dithiothreitol. In order to maintain a 2:1 (peptide:Hemin) stoichiometric ratio necessary for production of the “Sandwich” type constructs, the ferriprotoporphyrin IX chloride (Hemin) concentration was reduced to 0.125 mM in the above reaction scenario. Reactions were performed at room temperature in a nitrogen layered, sealed flask overnight and protected from light. After completion of the reaction, the reaction mixture was immediately frozen in a dry ice/acetone bath and lyophilized to eliminate the solvents. Next, the lyophilized powder containing crude microperoxidase along with reaction intermediates was dissolved in 15 ml degassed/deionized water and pelleted using ultracentrifugation in order to remove residual unreacted Hemin along with other insoluble products. Alternatively, the crude microperoxidase solution could be pre-cleared of salts and low MW reaction intermediates with flash chromatography using G10 Sephadex prior to reverse phase purification efforts. Supernatant fractions were separated from insoluble pellet and then loaded onto a preparative C-18 RP-HPLC (150×21.5 mm column) and purified using methods described above for the unconjugated peptide analogues. This was followed by a second round of lyophilization to obtain the purified microperoxidases in dry powder form. Verification of the exact mass was accomplished by MALDI-TOF analysis using an Applied Biosystems Voyager workstation and 2,5-dihydroxybenzoic acid matrix conditions.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A synthetic heme-containing molecule, comprising a heme group bound to:
two non-contiguous peptides each having an amino acid sequence of the formula (X)₁₋₂₀C(X)₁₋₂₀ where each X is independently any natural or non-canonical amino acid, and C represents a cysteine residue, wherein the cysteine residue of each peptide is bound to the heme group; or
a single contiguous peptide having the formula (X)₁₋₂₀C(X)₁₋₅C(X)₁₋₂₀ (SEQ ID NO: 3) where each X is independently any natural or non-canonical amino acid, and C represents a cysteine residue, wherein two cysteine residues of the peptide are bound to the heme group.

2. The synthetic heme-containing molecule of claim 1, comprising a heme group bound to two non-contiguous peptides, wherein the two non-contiguous peptides each have an amino acid sequence of the formula (X)₁₋₂₀CH(X)₁₋₁₉ (SEQ ID NO: 1) where each X is independently any natural or non-canonical amino acid, C represents a cysteine residue and H represent a histidine residue.

3-4. (canceled)

5. The synthetic heme-containing molecule of claim 1, comprising a heme group bound to a single contiguous peptide, wherein the peptide has an amino acid sequence of the formula (X)₁₋₂₀C(X)₁₋₅CH(X)₁₋₁₉ (SEQ ID NO: 4) where each X is independently any natural or non-canonical amino acid, C represents a cysteine residue and H represents a histidine residue.

6. (canceled)

7. The synthetic heme-containing molecule of claim 1, comprising a heme group bound to a single contiguous peptide, wherein the peptide has an amino acid sequence of the formula VQXCAQCX₁TVE (SEQ ID NO: 6) where X and X₁ are each independently any natural or non-canonical amino acid.

8. (canceled)

9. The synthetic heme-containing molecule of claim 1, comprising a heme group bound to two non-contiguous peptides, wherein the amino acid sequence of at least one of the two non-contiguous peptides comprises or consists of RCHGGR (SEQ ID NO: 11), or GCHGGD (SEQ ID NO: 12).

10. (canceled)

11. The synthetic heme-containing molecule of claim 1, comprising a heme group bound to a single contiguous peptide, wherein the amino acid sequence of the peptide comprises or consists of QHGCGGCHG (SEQ ID NO: 13), QHGCGGCGHG (SEQ ID NO: 14), or QHGGCGGCHG (SEQ ID NO: 15).

12. The synthetic heme-containing molecule of claim 1, wherein the peptide comprises at least one modification.

13. The synthetic heme-containing molecule of claim 12, wherein the at least one modification comprises a N-terminal modification, a C-terminal modification, or both N-terminal and C-terminal modifications.

14-15. (canceled)

16. The synthetic heme-containing molecule of claim 1, wherein the heme group is a microperoxidase.

17. (canceled)

18. The synthetic heme-containing molecule of claim 1, wherein the heme group is a metal porphyrin.

19-20. (canceled)

21. The synthetic heme-containing molecule of claim 1, wherein the heme group is a porphyrin with a vinyl group at carbon C3, C8, or both C3 and C8.

22. The synthetic heme-containing molecule of claim 1, wherein the heme group is a heme A, heme B, heme C, heme D, heme O, heme I, heme m, or heme S.

23. A method of removing carbon monoxide from hemoglobin in blood or animal tissue, comprising contacting the blood or animal tissue with the synthetic heme-containing molecule of claim 1.

24-27. (canceled)

28. A method of treating carboxyhemoglobinemia, cyanide poisoning or hydrogen sulfide (H₂S) poisoning in a subject, comprising administering to the subject the synthetic heme-containing molecule of claim 1.

29. The method of claim 28, wherein the method is a method of treating carboxyhemoglobinemia comprising:
selecting a subject with carboxyhemoglobinemia; and
administering to the subject a synthetic heme-containing molecule comprising a heme group bound to:
two non-contiguous peptides each having an amino acid sequence of the formula (X)₁₋₂₀C(X)₁₋₂₀ where each X is independently any natural or non-canonical amino acid, and C represents a cysteine residue, wherein the cysteine residue of each peptide is bound to the heme group; or
a single contiguous peptide having the formula (X)₁₋₂₀C(X)₁₋₅C(X)₁₋₂₀ (SEQ ID NO: 3) where each X is independently any natural or non-canonical amino acid, and C represents a cysteine residue, wherein two cysteine residues of the peptide are bound to the heme group.

30-31. (canceled)

32. The method of claim 23, wherein contacting the blood or animal tissue with the synthetic heme-containing molecule comprises administering a therapeutically effective amount of the synthetic heme-containing molecule to a subject, and wherein the synthetic heme-containing molecule is administered to the subject by intravenous infusion.

33. The method of claim 23, wherein contacting the blood or animal tissue with the synthetic heme-containing molecule comprises administering a therapeutically effective amount of the synthetic heme-containing molecule to a subject, and wherein the synthetic heme-containing molecule is administered at a dose of about 0.1 gram to about 300 grams.

34. The method of claim 33, wherein the synthetic heme-containing molecule is administered at a dose of about 1 gram to about 200 grams.

35. The method of claim 33, wherein the synthetic heme-containing molecule is administered at a dose of about 10 grams to about 100 grams.

36. The method of claim 33, wherein the synthetic heme-containing molecule is administered at a dose of about 30 grams to about 150 grams.
